(19) United States
(12) Patent Application Publication
(10) Pub. No.: US 2023/0021964 A1
(43) Pub. Date: Jan. 26, 2023

(54) ELECTRICAL STIMULATION THERAPY APPARATUS FOR DYSPHAGIA AND METHOD THEREOF

(71) Applicant: RS REHAB CO., LTD., Seongnam-si (KR)

(72) Inventors: Ju Seok RYU, Seongnam-si (KR); Jun Chang LEE, Seoul (KR); Jin Young KO, Hwaseong-si (KR)

(21) Appl. No.: 17/868,176

(22) Filed: Jul. 19, 2022

(30) Foreign Application Priority Data
Jul. 23, 2021 (KR) 10-2021-0097255

(51) Int. Cl. A61N 1/36; A61N 1/05

(52) U.S. Cl. CPC A61N 1/36003; A61N 1/0517

(57) ABSTRACT

Provided is an electrical stimulation therapy apparatus for dysphagia including an input unit configured to receive user information including a food type divided into a low-viscosity liquid and a high-viscosity liquid, a stimulation unit including a plurality of channels and configured to stimulate swallowing muscles of a user, and a processor configured to generate a control command for driving the stimulation unit, wherein the processor sequentially stimulates the swallowing muscles including a bilateral suprahyoid muscle (SH), a thyrohyoid muscle (TH), and a sternothyroid muscle (StH) of the user according to a preset muscle activation sequence based on the user information.

### TECHNICAL FIELD

The present disclosure relates to an electrical stimulation therapy apparatus for dysphagia and an electrical stimulation therapy method for dysphagia.

### BACKGROUND

Dysphagia makes swallowing difficult when eating or drinking. Furthermore, there are many cases of giving up food intake or nutritional intake in the mouth due to dysphagia, which is a big problem in terms of quality of life (QOL). At the same time, mental and physical stress is too great for a person who takes care of eating and drinking of a patient with dysphagia. In addition, a rate of pneumonia as causes of deaths of the elderly rapidly increases, but most of the causes of deaths are considered aspiration pneumonia due to dysphagia.

Symptoms of dysphagia occur in various diseases such as cerebrovascular disorders, Parkinson's disease, aging, dementia, head and neck cancer, radiation therapy, and cerebral palsy. In addition, the dysphagia may occur due to various causes, such as abnormalities in muscles and nerves of parts involved in swallowing, such as the oral cavity, pharynx, and esophagus, and abnormalities in the central nervous system that control the muscles and nerves. Therefore, improvement of the swallowing reflex is very important in improving QOL of people with dysphagia who are forced to eat and drink inconveniently and reducing a burden of nursing or in preventing aspiration pneumonia.

As a known therapy for dysphagia, various rehabilitation therapy methods have been implemented. However, there are cases where an effect of rehabilitation therapy is large or not great depending on people, and there are cases where cooperation of the people is required. Therefore, in order to increase effectiveness of rehabilitation therapy, a target person that may have the greatest effect of therapy techniques needs to be selected, and various therapy techniques with high therapeutic effects need to be developed.

In this regard, Korean Patent Registration No. 10-1798365 (title of invention: ELECTRICAL STIMULATION APPARATUS FOR DYSPHAGIA) discloses an electrical stimulation apparatus that is attached to a thyrohyoid muscle of a human body to detect time of swallowing food and applies electrical stimulation to bilateral digastric muscles of the human body.

### SUMMARY

Embodiments of the present disclosure provide an electrical stimulation therapy apparatus for dysphagia and an electrical stimulation therapy method that sequentially stimulate swallowing muscles of a user according to a preset muscle activation sequence based on user information of a food type and a muscle contraction type.

However, Technical problems to be solved by the present embodiments are not limited to the technical problems described above, and other technical problems may further exist.

According to an aspect of the present disclosure, an electrical stimulation therapy apparatus for dysphagia includes an input unit configured to receive user information including a food type divided into a low-viscosity liquid and a high-viscosity liquid, a stimulation unit including a plurality of channels and configured to stimulate swallowing muscles of a user, and a processor configured to generate a control command for driving the stimulation unit, wherein the processor sequentially stimulates the swallowing muscles including a bilateral suprahyoid muscle (SH), a thyrohyoid muscle (TH), and a sternothyroid muscle (StH) of the user according to a preset muscle activation sequence based on the user information.

According to another aspect of the present disclosure, an electrical stimulation therapy method performed by an electrical stimulation therapy apparatus for dysphagia includes receiving user information including a food type divided into a low-viscosity liquid and a high-viscosity liquid from a user through an input unit, and sequentially stimulating, by using a stimulation unit, a bilateral suprahyoid muscle (SH), a thyrohyoid muscle (TH), and a sternothyroid muscle (StH) of the user according to a preset muscle activation sequence based on the user information.

According to an embodiment of the present disclosure, the present disclosure has electrical stimulation therapy apparatus configured with three or more channels that can sequentially stimulate all muscles required for a swallowing process.

Furthermore, it is possible to induce contraction of sequentially swallowing muscle according to a normal swallowing muscle contraction pattern. Accordingly, the present disclosure has the effect of solving a problem that the known electrical stimulation therapy apparatus not sequentially stimulate swallowing muscles, but simultaneously contracts swallowing muscles, and does not reflect a normal swallowing muscle contraction pattern.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 is a configuration diagram of an electrical stimulation therapy apparatus for dysphagia according to an embodiment of the present disclosure;

FIG. 2 is a configuration diagram illustrating a configuration of a stimulation unit according to an embodiment of the present disclosure;

FIGS. 3A, 3B, 3C and 3D are example views illustrating a position at which a stimulation unit is installed, according to an embodiment of the present disclosure;

FIG. 4 illustrates a muscle contraction pattern of a swallowing muscle, according to an embodiment of the present disclosure;

FIGS. 5A and 5B illustrate two types of muscle contraction patterns according to an embodiment of the present disclosure;

FIG. 6A is an example diagram illustrating displacement of muscles over time while a user swallows a low-viscosity liquid, according to an embodiment of the present disclosure;

FIG. 6B is an example diagram illustrating displacement of muscles over time while a user swallows a high-viscosity liquid, according to an embodiment of the present disclosure; and

FIG. 7 is a flowchart illustrating an electrical stimulation therapy method of an electrical stimulation therapy apparatus for dysphagia, according to an embodiment of the present disclosure.

### DETAILED DESCRIPTION OF THE EMBODIMENTS

Hereinafter, embodiments of the present disclosure will be described in detail with reference to the accompanying drawings such that those skilled in the art may easily implement the present disclosure. However, the present disclosure may be embodied in several different forms and is not limited to the embodiments described herein. In order to clearly describe the present disclosure in the drawings, parts irrelevant to the description are omitted, and similar reference numerals are attached to similar elements throughout the specification.

Throughout the specification, when a portion is “connected” to another portion, this includes not only a case of being “directly connected” but also a case of being “electrically connected” with another element interposed therebetween. In addition, when a portion “includes” a certain component, this means that other components may be further included therein, rather than excluding the other components, unless otherwise stated, and it should be understood that a possibility of addition or existence of one or more other features, numbers, steps, operations, configuration elements, components, or combinations thereof is not precluded.

In the present specification, a “portion” includes a unit implemented by hardware, a unit implemented by software, and a unit implemented by using both. In addition, one unit may be implemented by using two or more pieces of hardware, and two or more units may be implemented by one piece of hardware. Meanwhile, “˜portion” is not limited to software or hardware, and “˜portion” may also be configured to be in an addressable storage medium or may also be configured to reproduce one or more processors. Thus, in one example, “˜portion” includes components such as software components, object-oriented software components, class components, and task components and includes processes, functions, properties, procedures, subroutines, segments of a program code, drivers, firmware, a microcode, a circuit, data, a database, data structures, tables, arrays, and variables. The functions provided in components and “˜portions” may be combined into a smaller number of components and “˜portions” or may be further separated into additional components and “˜portions”. In addition, components and “˜portions” may also be implemented to play one or more central processing units (CPUs) in a device.

A “network” refers to a connection structure capable of exchanging information between respective nodes such as terminals and devices, and includes a local area network (LAN), a wide area network (WAN), the Internet (WWW: world wide web), wired and wireless data communication networks, a telephone network, wired and wireless television networks, and so on. Wireless data communication networks include, for example, 3rd generation (3G), 4G, 5G, 3rd generation partnership project (3GPP), long term evolution (LTE), world interoperability for microwave access (WIMAX), Wi-Fi, Bluetooth communication, infrared communication, ultrasound communication, visible light communication (VLC), LiFi, and so on, but are not limited thereto.

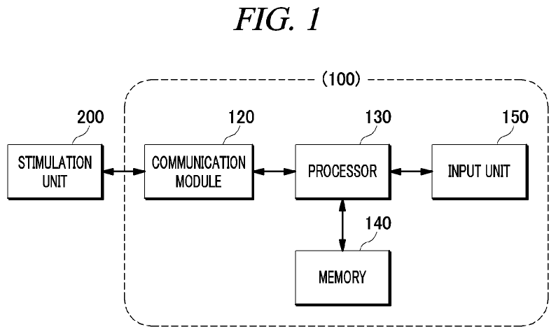

FIG. 1 is a configuration diagram illustrating an electrical stimulation therapy apparatus for dysphagia according to an embodiment of the present disclosure.

As illustrated in FIG. 1, an electrical stimulation therapy apparatus 100 for dysphagia according to an embodiment of the present disclosure includes an input unit 150 for receiving user information including food types classified into a low-viscosity liquid and a high-viscosity liquid, a stimulation unit 200 that includes a plurality of channels and stimulates a user's swallowing muscle, and a processor 130 that generates a control command for driving the stimulation unit 200, and the processor 130 sequentially stimulates swallowing muscles including bilateral suprahyoid muscles (SH), a thyrohyoid muscle (TH), and a sternothyroid muscle (StH) of a user according to a preset muscle activation sequence based on the user information. In addition, the user information further includes a muscle contraction type, and the input unit 150 may further receive a muscle contraction type other than a food type as the user information.

Therefore, the present disclosure has an effect of solving a problem that the known electrical stimulation therapy apparatus simultaneously contracts swallowing muscles, and sequentially stimulating all muscles required for a swallowing process.

Specifically, as illustrated in FIG. 1, the electrical stimulation therapy apparatus 100 includes a communication module 120, the processor 130, a memory 140, the input unit 150, and the stimulation unit 200.

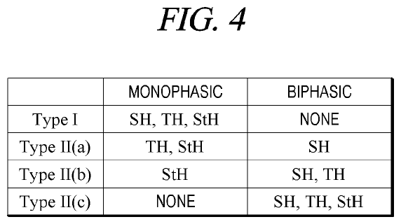

The input unit 150 may be configured to select user information through a physical button or a touch input on a display. Exemplarily, a user may select one of a low-viscosity liquid such as water or a high-viscosity food with viscosity, such as yogurt through the input unit 150 and may select a muscle contraction pattern described below with reference to FIG. 4.

The communication module 120 performs data communication with the stimulation unit 200. The communication module 120 provides a communication interface necessary to provide signals transmitted to and received from respective units through a communication network in the form of packet data. Here, the communication module 120 may include hardware and software necessary for transmitting and receiving signals such as control signals or data signals through wired/wireless connection with other network devices.

The memory 140 may store a program for controlling the stimulation unit 200, and the program for controlling the stimulation unit 200 stored in the memory 140 may be driven by the processor 130.

In addition, the memory 140 performs a function of temporarily or permanently storing data processed by the processor 130. Here, the memory 140 may include a volatile storage medium or a non-volatile storage medium, but the scope of the present disclosure is not limited thereto.

The memory 140 may store a separate program such as an operating system for processing and controlling the processor 130 or may perform a function of temporarily storing input data or output data.

The memory 140 may include at least one type of storage medium among a flash memory type, a hard disk type, a multimedia card micro type, a card type memory (for example, a secure digital (SD) memory or an extreme digital (XD) memory), and a random access memory (RAM), and a read-only memory (ROM). In addition, a user terminal may operate a web storage that performs a storage function of the memory 140 on the Internet.

The processor 130 executes a program for controlling the stimulation unit 200 stored in the memory 140 and controls the overall operation for controlling the electrical stimulation therapy apparatus for dysphagia.

To this end, the processor 130 may include one or more processing units (a central processing unit (CPU), a micro-processor, a digital signal processor (DSP), and so on), RAM, ROM, and so on, and may read a program stored in the memory 140 into the RAM and execute the program by using the one or more processing units. In addition, according to an embodiment, a term “processor” may be interpreted as the same meaning as a terms such as a “controller”, an “arithmetic unit”, or a “controller”.

A schematic procedure of controlling an electrical stimulation therapy apparatus for dysphagia through a program for controlling the stimulation unit 200 according to an embodiment of the present disclosure is as follows.

The processor 130 may control the stimulation unit 200 such that swallowing muscles including bilateral suprahyoid muscles (SH), a thyrohyoid muscle (TH), and a sternothyroid muscle (StH) of a user are sequentially stimulated according to execution of a program and a preset muscle activation sequence, based on the user information input to the input unit 150.

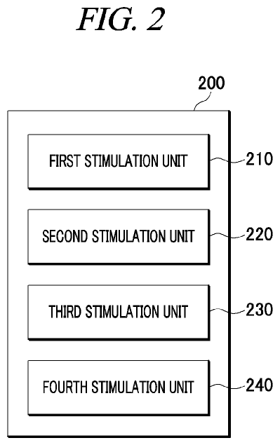

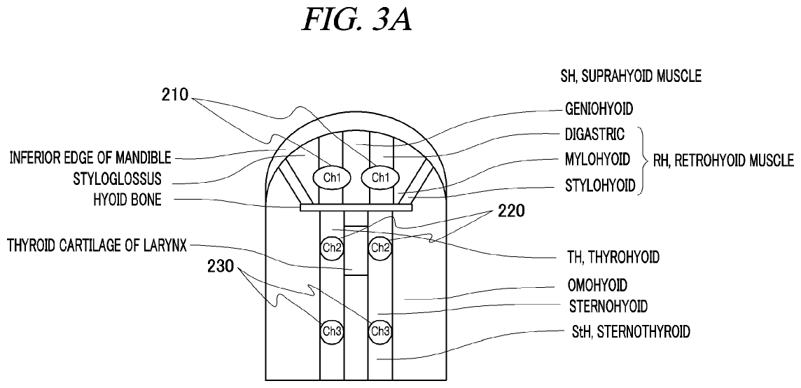

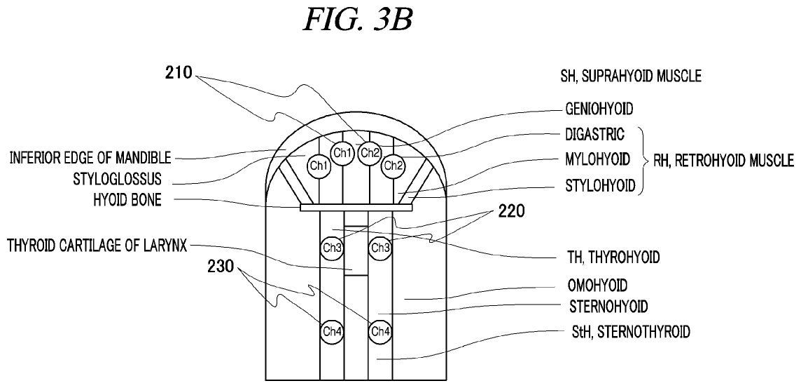

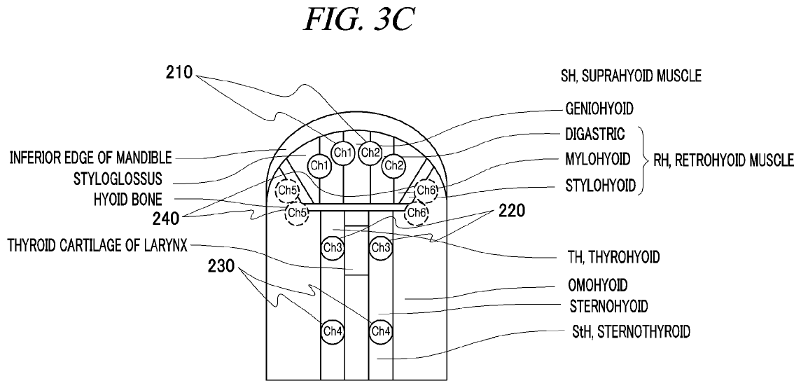

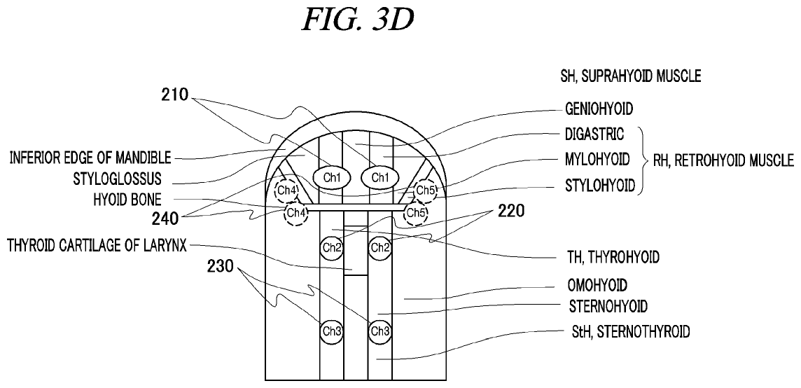

FIG. 2 is a configuration diagram illustrating a configuration of the stimulation unit according to an embodiment of the present disclosure. FIGS. 3A, 3B, 3C and 3D are example views illustrating a position at which the stimulation unit is installed, according to an embodiment of the present disclosure.

As illustrated in FIG. 2, the stimulation unit 200 includes a first stimulation unit 210 attached to digastric and mylohyoid to stimulate bilateral suprahyoid muscles (SH), a second stimulation unit 220 attached onto both sides of thyroid cartilage to stimulate a thyrohyoid (TH) muscle, and a third stimulation unit 230 attached to an inner side of sternocleidomastoid muscle and to a lower portion of the thyroid cartilage to stimulate a sternohyoid (StH) muscle.

For example, the processor 130 may sequentially drive the first stimulation unit 210 to the third stimulation unit 230 to stimulate a user's swallowing muscle according to a preset muscle activation sequence.

Illustratively, the stimulation unit 200 may be configured with three channels or four channels. For example, the channel means a path through which a current flows when a closed circuit including a power source is formed and a current flows through the closed circuit. That is, one channel may include a power source, two electrodes (anode and cathode) connected to the power source, and a muscle connecting the electrodes to each other. In this case, the channel is used to give electrical stimulation, and each channel may include two electrodes (anode and cathode).

Referring to FIG. 3A, the stimulation unit 200 may include, for example, three channels. For example, the first stimulation unit 210 may include a first channel Ch1 that stimulates digastric (venter anterior) and mylohyoid as targets. In this case, two electrodes of the first channel Ch1 may be respectively arranged on right and left sides of the bilateral suprahyoid muscles (SH). In addition, as illustrated in FIG. 3A, when the bilateral suprahyoid muscles (SH) are stimulated by the first channels Ch1, it is effective that pads of the first channel Ch1 have larger areas than pads of a second channel and a third channel so as to cover the two muscles (the digastric and the mylohyoid). In this case, electrodes of the first channel Ch1 may be separated from each other by about 1 cm but are not limited thereto.

The second stimulation unit 220 may include a second channel Ch2 that stimulates a thyrohyoid muscle (TH) as a target. In this case, electrodes of the second channel Ch2 may be arranged on upper portions of both sides of the thyroid cartilage.

The third stimulation unit 230 may include a third channel Ch3 that stimulates sternohyoid, omohyoid, and a sternothyroid (StH) muscle as targets. In this case, electrodes of the third channel Ch3 may be respectively arranged above the sternocleidomastoid muscle and below the thyroid cartilage.

In another example, as illustrated in FIG. 3B, the stimulation unit 200 may include four channels. For example, the first stimulation unit 210 may include the first channel Ch1 and the second channel Ch2 that stimulate stimulates the digastric (venter anterior) and the mylohyoid as targets. In this case, electrodes of the first channel Ch1 may be arranged on the right suprahyoid muscle (SH), and electrodes of the second channel Ch2 may be arranged on the left suprahyoid muscle (SH). Here, the electrodes of each channel may be located above a hyoid bone at an interval of 1 cm and arranged behind jaw, but the present disclosure is not limited thereto, and when the chin is too narrow depending on a human face shape, the electrodes may be arranged with an interval narrower than 1 cm.

The second stimulation unit 220 may include a third channel Ch3 that stimulates the thyrohyoid muscle (TH) as a target. In this case, electrodes of the third channel Ch3 may be respectively arranged at upper portions of both sides of the thyroid cartilage.

The third stimulation unit 230 may include a fourth channel Ch4 that stimulates sternohyoid, omohyoid, and a sternothyroid (StH) muscle as targets. In this case, electrodes of the fourth channel Ch4 may be respectively arranged above the sternocleidomastoid muscle and below the thyroid cartilage.

In a further embodiment, as illustrated in FIG. 3C, the stimulation unit 200 may also include six channels, and the stimulation unit 200 may further include a fourth stimulation unit 240 in addition to the first to third stimulation units 210 to 230 illustrated in FIG. 3B. For example, the fourth stimulation unit 240 may include a fifth channel Ch5 and a sixth channel Ch6 that stimulate superior pharyngeal constrictor of a posterior portion of the digastric and styloglossus (a retrohyoid muscle (RH)) as a target. In this case, electrodes of the fifth channel Ch5 may be respectively arranged on the right superior pharyngeal constrictors (preferably, just behind the jaw and in front of the sternocleidomastoid muscle), and electrodes of the sixth channel Ch6 may be respectively arranged on the left superior pharyngeal constrictors. In another example, as illustrated in FIG. 3D, the stimulation unit 200 may also include five channels and may further include the fourth stimulation unit 240 in addition to the first to third stimulation units 210 to 230 illustrated in FIG. 3A. For example, the fourth stimulation unit 240 may include the fourth channel Ch4 and the fifth channel Ch5 that stimulate superior pharyngeal constrictor of a posterior portion of the digastric and styloglossus (a retrohyoid muscle (RH)) as a target. In this case, electrodes of the fourth channel Ch4 may be respectively arranged on the right superior pharyngeal constrictors (preferably, just behind the jaw and in front of the sternocleidomastoid muscle), and electrodes of the fifth channel Ch5 may be respectively arranged on the left superior pharyngeal constrictors. In addition, stimulating the pharyngeal constrictor by the fourth stimulation unit 240 according to a muscle activation sequence at a point in time when the second stimulation unit 220 stimulates the thyrohyoid muscle (TH) may help a swallowing process, but the stimulation point in time is not limited thereto.

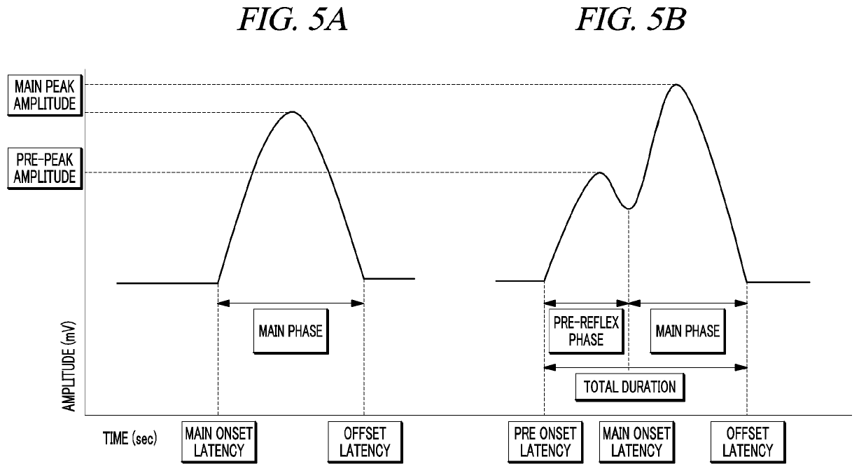

FIG. 4 illustrates a muscle contraction pattern of a swallowing muscle, according to an embodiment of the present disclosure. FIG. 5 illustrates two types of muscle contraction patterns according to an embodiment of the present disclosure.

As illustrated in FIG. 5, a muscle contraction pattern (a muscle contraction type) includes a first type (Type I) in which all the swallowing muscles have a monophasic pattern, and a second type (Type II) in which at least one of the swallowing muscles has a biphasic pattern and the rest of the swallowing muscles have a monophasic pattern.

As illustrated in (a) of FIG. 5, the monophasic pattern represents only one main peak amplitude, and as illustrated in (b) of FIG. 5, the biphasic pattern represents two or more prior peak amplitudes and a main peak amplitude.

For example, referring to FIG. 4, the first type (Type I) includes a first pattern in which all of the suprahyoid muscle (SH), the thyrohyoid muscle (TH), and the sternothyroid muscle (StH) have a monophasic pattern.

The second type (Type II) includes a second pattern (Type II(a)) in which the thyrohyoid muscle (TH) and the sternothyroid muscle (StH) have a monophasic pattern and the suprahyoid muscle (SH) has a biphasic pattern, a third pattern (Type II(b)) in which the sternothyroid muscle (StH) has a monophasic pattern and the suprahyoid muscle (SH) and the thyrohyoid muscle (TH) have a biphasic pattern, and a fourth pattern (Type II(c)) in which all of the suprahyoid muscle (SH), the thyrohyoid muscle (TH), and the sternothyroid muscle (StH) have a biphasic pattern.

For example, a user may input a muscle contraction type through the input unit 150. For example, the input unit 150 may include buttons, a touch input, or so on. The user may select the first type (the first pattern) or the second type (the second pattern to fourth pattern) according to a criterion.

In one embodiment, in order to describe a criterion for inputting the muscle contraction type, a user (patient) may swallow food a predetermined number of times in a state in which electrodes are attached to the user's swallowing muscles. In this case, the processor 130 may collect and record a muscle activation sequence by using pads of electrodes of channels respectively attached to the user's swallowing muscles. Thereafter, the processor 130 may determine whether a pattern of each swallowing muscle is monophasic or biphasic (polyphasic) based on the collected muscle activation sequence and may provide a muscle contraction type to the user. Thereafter, when the user inputs his/her muscle contraction type through the input unit 150, the processor 130 may stimulate the swallowing muscle through the stimulation unit 200 at the timing when the user swallows food. That is, the processor 130 may sequentially stimulate all the swallowing muscles according to the muscle activation sequence corresponding to the user's muscle contraction type.

In another embodiment, when a user inputs a stimulus through the input unit 150 while swallowing food for a certain duration of time, the processor 130 may stimulate each swallowing muscle through the stimulation unit 200. In this case, the processor 130 may learn and store the timing at which the user inputs the stimulus, determine the muscle activation sequence based on the stimulus input timing, and provide the muscle contraction type to the user.

For example, in a case of the second type, when only a starting point is matched through training, contraction sequences for each muscle may be the same as each other, thereby being applied to a current stimulation protocol.

For example, when a user selects the first type, the processor 130 may drive the first stimulation unit 210 to the third stimulation unit 230 according to a muscle activation sequence corresponding to the first pattern (Type I).

In another example, when a user selects the second type (a second pattern to a fourth pattern), the processor 130 may drive the first stimulation unit 210 to the third stimulation unit 230 according to a muscle activation sequence corresponding to the selected second pattern (Type II(a)) to fourth pattern (Type II(c)).

In addition, a user may input a food type through the input unit 150. For example, the type of food that the user wants to eat may include a low-viscosity liquid or a high-viscosity liquid. In this case, a criterion for selecting the food type may include a high-viscosity liquid when the user (patient) chews a solid such as rice well and may include a low-viscosity liquid when the user swallows only a liquid such as water.

Specifically, even when the same type (pattern) is selected according to the food type selected by a user, times when the first stimulation unit 210 to the third stimulation unit 230 stimulate the bilateral suprahyoid muscles (SH), the thyrohyoid muscle (TH), and the sternothyroid muscle (StH) are different from each other. That is, even when the types (patterns) are the same as each other, stimulation points in time at which each stimulation unit 200 stimulates target muscles for each food type (a low-viscosity liquid or a high-viscosity liquid) input by a user are different from each other.

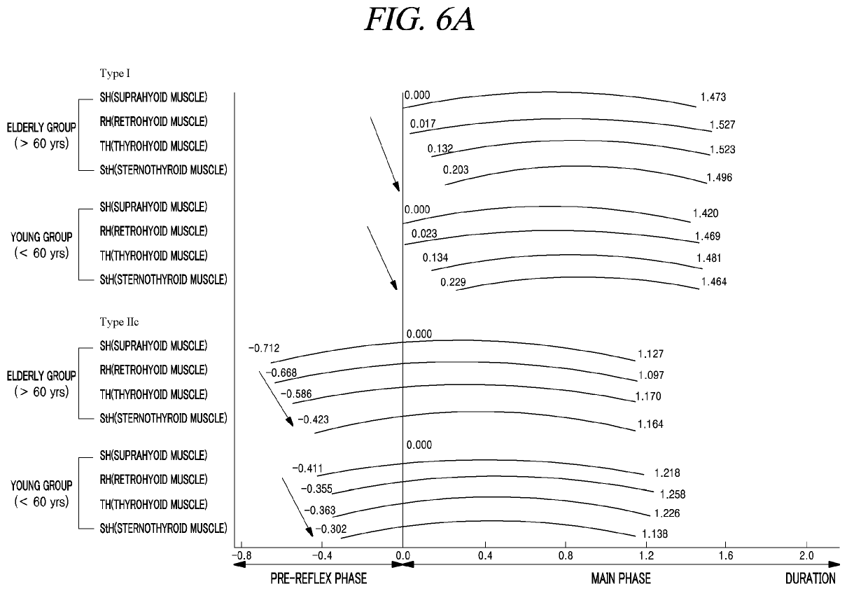

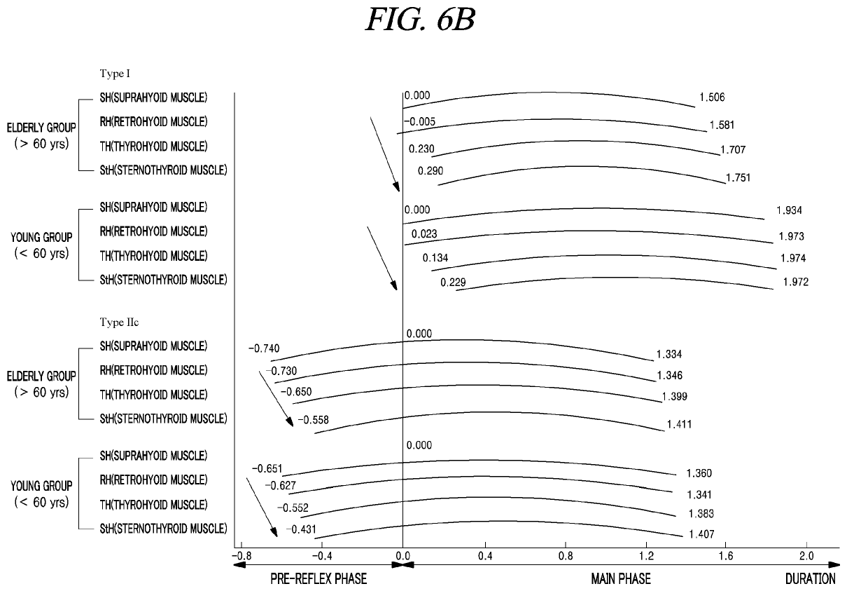

FIG. 6A is an example diagram illustrating displacement of muscles over time while a user swallows a low-viscosity liquid, according to an embodiment of the present disclosure. FIG. 6B is an example diagram illustrating displacement of muscles over time while a user swallows a high-viscosity liquid, according to an embodiment of the present disclosure.

FIGS. 6A and 6B illustrate results of measuring electromyogram signals for a healthy elderly group (>60 years old) and a young group (<60 years old) while swallowing a low-viscosity liquid of 5 cc and a high-viscosity liquid of 5 cc by using 6-channel surface electrodes (see FIG. 3C) arranged over the bilateral suprahyoid muscles (SH), a bilateral retrohyoid muscle (RH), the thyrohyoid muscle (TH), and the sternothyroid muscle (StH). That is, FIGS. 6A and 6B illustrate that contraction is made in the order of the bilateral suprahyoid muscles (SH), the thyrohyoid muscle (TH), and the sternothyroid muscle (StH) in both groups in which a swallowing motion of a contraction pattern (a muscle activation sequence) of a swallowing muscle is healthy. Accordingly, the processor 130 may induce contraction of a user's swallowing muscle according to a normal muscle activation sequence based on the type of muscle contraction and a food type input by the user.

Hereinafter, an example of stimulation timing of each stimulation unit 200 for each food type is described with reference to FIGS. 6A and 6B by comparing the first type (Type I) with the second type (Type II(c)).

For example, a muscle activation sequence of the first type (first pattern) includes a main phase (that is, all swallowing muscles have a monophasic pattern). That is, referring to FIG. 6A, in the main phase, when an elderly group (>60 years old) inputting the first type swallows a low-viscosity liquid or so on, the first stimulation unit 210 may stimulate the suprahyoid muscle (SH), and after 0.120 s to 0.150 s based on the start of contraction of the suprahyoid muscle (SH), the second stimulation unit 220 may stimulate the thyrohyoid muscle (TH), and after 0.150 s to 0.250 s, the third stimulation unit 230 may stimulate the sternothyroid muscle (StH). When young and old people swallow a low-viscosity liquid and so on, a difference in muscle contraction pattern is small, and thus, similar electrical stimulation protocol may be applied to the first type. Accordingly, contraction of a user's swallowing muscle may be induced in an order of the bilateral suprahyoid muscles (SH), the thyrohyoid muscle (TH), and the sternothyroid muscle (StH) according to a normal muscle activation sequence. In addition, the retrohyoid muscle (RH) illustrated in FIG. 6A is eccentrically contracted when the bilateral suprahyoid muscles (SH) contract, and thus, a muscles related to the retrohyoid muscle (RH) need not be stimulated. That is, when the retrohyoid muscle (RH) is stimulated after 0.010 s to 0.025 s as soon as the suprahyoid muscle (SH) is stimulated, passage of bolus may be prevented, and a swallowing process is rather hindered.

In addition, referring to FIG. 6B, in the main phase, while an elderly group (>60 years old) inputting the first type swallows a high-viscosity liquid, the first stimulation unit 210 may stimulate the suprahyoid muscle (SH), and after 0.200 s to 0.250 s based on the start of contraction of the suprahyoid muscle (SH), the second stimulation unit 220 may stimulate the thyrohyoid muscle (TH), and after 0.250 s to 0.300 s, the third stimulation unit 230 may stimulate the sternothyroid muscle (StH).

In addition, a young group (<60 years old) has a sequence in which contraction of the swallowing muscle starts, that is, an order of the bilateral suprahyoid muscles (SH), the thyrohyoid muscle (TH), and the sternothyroid muscle (StH), which is similar to the elderly group, but when the young group swallows a high-viscosity liquid, each muscle maintains contraction longer to induce strong swallowing. That is, an electrical stimulation sequence and a contraction duration are the most effective and safe order in a normal state (young adult).

In addition, the elderly group has a greater time interval for sequential contraction in the young group and has a reduced ability (sensory or cognitive function in the oral cavity) to sense viscosity, and thus, there is no difference in contraction durations of the low-viscosity liquid and the high-viscosity liquid. That is, a muscle contraction duration of the elderly group is shorter than a muscle contraction duration of the young group.

Therefore, when the elderly group swallows a high-viscosity liquid, stimulation is required for the elderly group to swallow the high-viscosity liquid for a long time by extending the contraction duration such that the elderly group swallows the high-viscosity liquid safely and effectively.

In a time interval for sequential stimulation of a swallowing muscle, high-viscosity food is difficult to be sucked due to a high viscosity, and a lot of residual low-viscosity liquid remains or is difficult to be swallowed. Therefore, the time interval for stimulating the swallowing muscle may be set to 0.200 s to 0.250 s during which a thyrohyoid muscle (TH) is stimulated and may be set to 0.250 s to 0.300 s during which the sternothyroid muscle (StH) is stimulated, as in the elderly group. In this case, there is no problem when the stimulation is made at the same time interval (as in low viscosity) as the young group, but such stimulation appears more comfortable and effective.

Accordingly, contraction of a user's swallowing muscle may be induced in an order of the bilateral suprahyoid muscles (SH), the thyrohyoid muscle (TH), and the sternothyroid muscle (StH) according to a normal muscle activation sequence. Similarly, the retrohyoid muscle (RH) illustrated in FIG. 6B is eccentrically contracted when the bilateral suprahyoid muscles (SH) contract, and thus, a muscles related to the retrohyoid muscle (RH) need not be stimulated at a corresponding point in time.

In addition, a muscle activation sequence of the second type (fourth pattern) includes a pre-reflex phase and a main phase (that is, all swallowing muscles have a biphasic pattern). That is, referring to FIG. 6A, in the pre-reflex phase (start time of the pre-reflex phase based on a start point (0 s) of the main phase is indicated in FIG. 6A) while the elderly group (>60 years old) inputting the second type swallows a low-viscosity liquid, the first stimulation unit 210 may stimulate the suprahyoid muscle (SH) at −0.720 ms to −0.680 ms before the main phase starts, and the second stimulation unit 220 may stimulate the thyrohyoid muscle (TH) at −0.650 ms to −0.550 ms, and the third stimulation unit 230 may stimulate the sternothyroid muscle (StH) at −0.450 ms to −0.400 ms. Accordingly, contraction of a user's swallowing muscle may be induced in an order of the bilateral suprahyoid muscles (SH), the thyrohyoid muscle (TH), and the sternothyroid muscle (StH) according to a normal muscle activation sequence.

In addition, referring to FIG. 6B, in the pre-reflex phase while the elderly group (>60 years old) inputting the second type swallows a high-viscosity liquid, the first stimulation unit 210 may stimulate the suprahyoid muscle (SH) at −0.750 ms to −0.730 ms before the main phase starts, and the second stimulation unit 220 may stimulate the thyrohyoid muscle (TH) at −0.700 ms to −0.600 ms, and the third stimulation unit 230 may stimulate the sternothyroid muscle (StH) at −0.600 ms to −0.550 ms. Accordingly, contraction of a user's swallowing muscle may be induced in an order of the bilateral suprahyoid muscles (SH), the thyrohyoid muscle (TH), and the sternothyroid muscle (StH) according to a normal muscle activation sequence.

In addition, the pre-reflex phase in the second type means protective contraction for preventing suction. That is, the elderly group does not extend a contraction duration for high-viscosity diet like young adults in the first type, but it may be seen that the elderly group effectively increases the contraction duration like young adults in the second type. Therefore, the pre-reflex phase in the second type appears as an adaptive mechanism for adaptation to swallowing in the elderly group.

The second type does not show a difference in a contraction sequence and a contraction duration of a muscle in terms of electrical stimulation (except for the pre-reflex phase), and thus, the same stimulation as the first type may be applied, but more training of a patient is required to synchronize the electrical stimulation with actual swallowing.

Hereinafter, descriptions of components performing the same function among the components described with reference to FIGS. 1 to 6B are omitted.

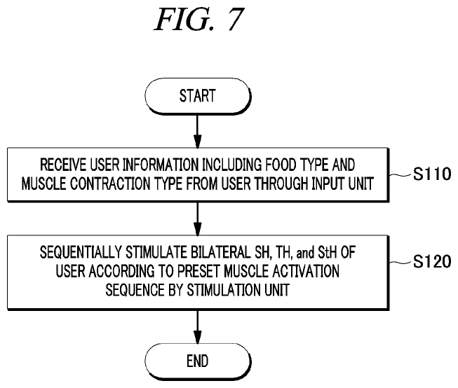

FIG. 7 is a flowchart illustrating an electrical stimulation therapy method of an electrical stimulation therapy apparatus for dysphagia, according to an embodiment of the present disclosure.

Referring to FIG. 7, an electrical stimulation therapy method performed by an electrical stimulation therapy apparatus for dysphagia, according to an embodiment of the present disclosure includes step S110 of receiving user information including a food type divided into a low-viscosity liquid and a high-viscosity liquid, from a user through the input unit 150, and step S120 of sequentially stimulating by using a stimulation unit 200, bilateral suprahyoid muscles (SH), a thyrohyoid muscle (TH), and a sternothyroid muscle (StH) of a user according to a preset muscle activation sequence based on the user information.

A muscle contraction type includes a first type in which all swallowing muscles have a monophasic pattern, and a second type in which at least one of the swallowing muscles has a biphasic pattern and the rest of the swallowing muscles have a monophasic pattern, and the monophasic pattern represents only one main peak amplitude, and the biphasic pattern represents two or more prior peak amplitudes and a main peak amplitude.

The first type includes a first pattern in which all of a suprahyoid muscle (SH), a retrohyoid muscle (RH), a thyrohyoid muscle (TH), and a sternothyroid muscle (StH) have a monophasic pattern, and the second type includes a second pattern in which the thyrohyoid muscle (TH) and the sternothyroid muscle (StH) have a monophasic pattern and the suprahyoid muscle (SH) and the retrohyoid muscle (RH) have a biphasic pattern, a third pattern in which the sternothyroid muscle (StH) has a monophasic pattern and the suprahyoid muscle (SH), the retrohyoid muscle (RH), and the thyrohyoid muscle (TH) have a biphasic pattern, and a fourth pattern in which all of the suprahyoid muscle (SH), the retrohyoid muscle (RH), the thyrohyoid muscle (TH), and the sternothyroid muscle (StH) have a biphasic pattern.

A stimulation unit includes a first stimulation unit attached to digastric and mylohyoid to stimulate bilateral suprahyoid muscles (SH), a second stimulation unit attached to the rear of the jaw and the front of the sternocleidomastoid muscle and attached onto both sides of thyroid cartilage to stimulate a thyrohyoid (TH) muscle, and a third stimulation unit attached to an inner side of sternocleidomastoid muscle and to a lower portion of the thyroid cartilage to stimulate a sternohyoid (StH) muscle.

An embodiment of the present disclosure may also be implemented in the form of a recording medium including instructions executable by a computer such as a program module executed by the computer. A computer-readable medium may be any available medium that may be accessed by a computer and include all of volatile and nonvolatile media and removable and non-removable media. In addition, the computer-readable medium may include a computer storage medium. The computer storage medium includes all of volatile and nonvolatile media and removable and non-removable media implemented by any method or technology for storing information such as computer readable instructions, data structures, program modules or other data.

Although the method and system of the present disclosure are described with reference to specific embodiments, some or all of their components or operations may be implemented by using a computer system having a general-purpose hardware architecture.

The above description of the present disclosure is for illustration, and those skilled in the art to which the present disclosure belongs will be able to understand that it may be easily modified into other specific forms without changing the technical idea or essential features of the present disclosure. Therefore, it should be understood that the embodiments described above are illustrative in all respects and not restrictive. For example, each component described as a single type may be implemented in a distributed form, and likewise components described as distributed may be implemented in a combined form.

The scope of the present disclosure is indicated by the following claims rather than the above detailed description, and all changes or modifications derived from the meaning and scope of the claims and their equivalent concepts should be interpreted as being included in the scope of the present disclosure.

According to an embodiment of the present disclosure, an electrical stimulation therapy apparatus including three or more channels may stimulate all muscles necessary for a process of swallowing (swallowing process).

In addition, muscle contraction may be sequentially induced according to a normal swallowing muscle contraction pattern. Accordingly, it is possible to solve a problem that the known electrical stimulation therapy machine does not perform sequential stimulation and a normal muscle contraction pattern is not reflected due to simultaneously swallowed muscle contract.

What is claimed is:

1. An electrical stimulation therapy apparatus for dysphagia, comprising:
an input unit configured to receive user information including a food type divided into a low-viscosity liquid and a high-viscosity liquid;
a stimulation unit including a plurality of channels and configured to stimulate swallowing muscles of a user; and
a processor configured to generate a control command for driving the stimulation unit,
wherein the processor sequentially stimulates the swallowing muscles including a bilateral suprahyoid muscle (SH), a thyrohyoid muscle (TH), and a sternothyroid muscle (StH) of the user according to a preset muscle activation sequence based on the user information.

2. The electrical stimulation therapy apparatus of claim 1, wherein
the user information further includes a muscle contraction type, and
the muscle contraction type includes a first type in which all the swallowing muscles have a monophasic pattern, and a second type in which at least one of the swallowing muscles has a biphasic pattern and the other swallowing muscles have a monophasic pattern.

3. The electrical stimulation therapy apparatus of claim 2, wherein
the first type includes a first pattern in which all of the suprahyoid muscle (SH), the thyrohyoid muscle (TH), and the sternothyroid muscle (StH) have a monophasic pattern, and
the second type includes a second pattern in which the thyrohyoid muscle (TH) and the sternothyroid muscle (StH) have a monophasic pattern and the suprahyoid muscle (SH) has a biphasic pattern, a third pattern in which the sternothyroid muscle (StH) has a monophasic pattern and the suprahyoid muscle (SH) and the thyrohyoid muscle (TH) have a biphasic pattern, and a fourth pattern in which all of the suprahyoid muscle (SH), the thyrohyoid muscle (TH), and the sternothyroid muscle (StH) have a biphasic pattern.

4. The electrical stimulation therapy apparatus of claim 1, wherein the stimulation unit includes a first stimulation unit attached to digastric and mylohyoid to stimulate bilateral suprahyoid muscles (SH), a second stimulation unit attached onto both sides of thyroid cartilage to stimulate a thyrohyoid (TH) muscle, and a third stimulation unit attached to an inner side of sternocleidomastoid muscle and to a lower portion of the thyroid cartilage to stimulate a sternohyoid (StH) muscle.

5. An electrical stimulation therapy method performed by an electrical stimulation therapy apparatus for dysphagia, the electrical stimulation therapy method comprising:
receiving user information including a food type divided into a low-viscosity liquid and a high-viscosity liquid from a user through an input unit; and
sequentially stimulating, by using a stimulation unit, a bilateral suprahyoid muscle (SH), a thyrohyoid muscle (TH), and a sternothyroid muscle (StH) of the user according to a preset muscle activation sequence based on the user information.

6. The electrical stimulation therapy method of claim 5, wherein
the user information further includes a muscle contraction type, and
the muscle contraction type includes a first type in which all the swallowing muscles have a monophasic pattern, and a second type in which at least one of the swallowing muscles has a biphasic pattern and the other swallowing muscles have a monophasic pattern.

7. The electrical stimulation therapy method of claim 6, wherein
the first type includes a first pattern in which all of the suprahyoid muscle (SH), the thyrohyoid muscle (TH), and the sternothyroid muscle (StH) have a monophasic pattern, and
the second type includes a second pattern in which the thyrohyoid muscle (TH) and the sternothyroid muscle (StH) have a monophasic pattern and the suprahyoid muscle (SH) has a biphasic pattern, a third pattern in which the sternothyroid muscle (StH) has a monophasic pattern and the suprahyoid muscle (SH) and the thyrohyoid muscle (TH) have a biphasic pattern, and a fourth pattern in which all of the suprahyoid muscle (SH), the thyrohyoid muscle (TH), and the sternothyroid muscle (StH) have a biphasic pattern.

8. The electrical stimulation therapy method of claim 5, wherein the stimulation unit includes a first stimulation unit attached to digastric and mylohyoid to stimulate bilateral suprahyoid muscles (SH), a second stimulation unit attached onto both sides of thyroid cartilage to stimulate a thyrohyoid (TH) muscle, and a third stimulation unit attached to an inner side of sternocleidomastoid muscle and to a lower portion of the thyroid cartilage to stimulate a sternohyoid (StH) muscle.
